(19) United States
(12) Patent Application Publication
(10) Pub. No.: US 2009/0252655 A1
(43) Pub. Date: Oct. 8, 2009

(54) PHOTO-CATALYST AIR CLEANER

(75) Inventors: CHIH-PENG HSU, HuKou (TW); CHUNG-MIN CHANG, HuKou (TW); TSE-AN LEE, HuKou (TW)

Correspondence Address: PCE INDUSTRY, INC.;ATT. Steven Reiss, 288 SOUTH MAYO AVENUE, CITY OF INDUSTRY, CA, 91789, US

(73) Assignee: ADVANCED OPTOELECTRONIC TECHNOLOGY, INC., Hsinchu Hsien (TW)

(21) Appl. No.: 12/251,719

(22) Filed: Oct. 15, 2008

(30) Foreign Application Priority Data
Apr. 2, 2008 (CN) 200810300825.X

(51) Int. Cl. A61L 9/00

(52) U.S. Cl. 422/122

(57) ABSTRACT

A photo-catalyst air cleaner includes at least one light source, a photo-catalyst filter and a first water supply. The photo-catalyst filter includes a hydrophilic substrate with a layer formed on a surface of the substrate exposed to light from the at least one light source. The water supply is configured for supplying water for absorption by the substrate, thereby humidifying surfaces of the photo-catalyst filter.

### CROSS-REFERENCE TO RELATED APPLICATIONS

This application is related to the following commonly-assigned copending applications: Ser. No. 12/240,422, entitled “AIR CLEANER” (attorney docket number US 18918). Disclosure of the above-identified application is incorporated herein by reference.

### BACKGROUND

1. Technical Field

The present invention generally relates to air cleaning, and particularly to a photo-catalyst air cleaner providing self-contained humidifying capability.

2. Description of Related Art

In recent years, photo-catalyst air cleaners have become widely used. Photo-catalysts, for example titanium oxide (TiO₂), are excited by photo-energy to sterilize microbes and decompose pollutants. In operation, the photo-catalysts is exposed to UV light, and electron-hole pairs are excited from within the photo-catalysts to the surface thereof, to react with the water vapor, forming hydroxyl radical (.OH) having higher oxidative ability. Pollutants are easily adsorbed by the water vapor on the surface. The photo-catalysts, when excited, kills microbes and decomposes organic pollutants into water vapor (H₂O) and carbon dioxide (CO₂). However, performance of the cleaners is dependent on the amount of water vapor present in the environment. In a dry setting, air cleaning performance is reduced.

Therefore, what is needed is a photo-catalyst air cleaner which can overcome the described limitations.

### SUMMARY

A photo-catalyst air cleaner includes at least one light source, a photo-catalyst filter, and a water supply. The photo-catalyst filter includes a hydrophilic substrate with a layer on a surface thereof, exposed to light from the at least one light source. The water supply supplies water for absorption by the substrate, thereby humidifying surfaces of the photo-catalyst filter.

Other advantages and novel features of the present photo-catalyst air cleaner will become more apparent from the following detailed description when taken in conjunction with the accompanying drawings, in which:

### BRIEF DESCRIPTION OF THE DRAWINGS

Many aspects of the present photo-catalyst air cleaner can be better understood with reference to the following drawings. The components in the drawings are not necessarily drawn to scale, the emphasis instead being placed upon clearly illustrating the principles of the present illumination device. Moreover, in the drawings, like reference numerals designate corresponding parts throughout the several views.

FIG. 1 is a schematic view of a photo-catalyst air cleaner, in accordance with a first embodiment.

FIG. 2 is an enlarged partial cross-section of a photo-catalyst filter of FIG. 1.

FIG. 3 is a schematic view of a photo-catalyst air cleaner, in accordance with a second embodiment.

FIG. 4 is a schematic view of a photo-catalyst air cleaner, in accordance with a third embodiment.

### DETAILED DESCRIPTION OF THE INVENTION

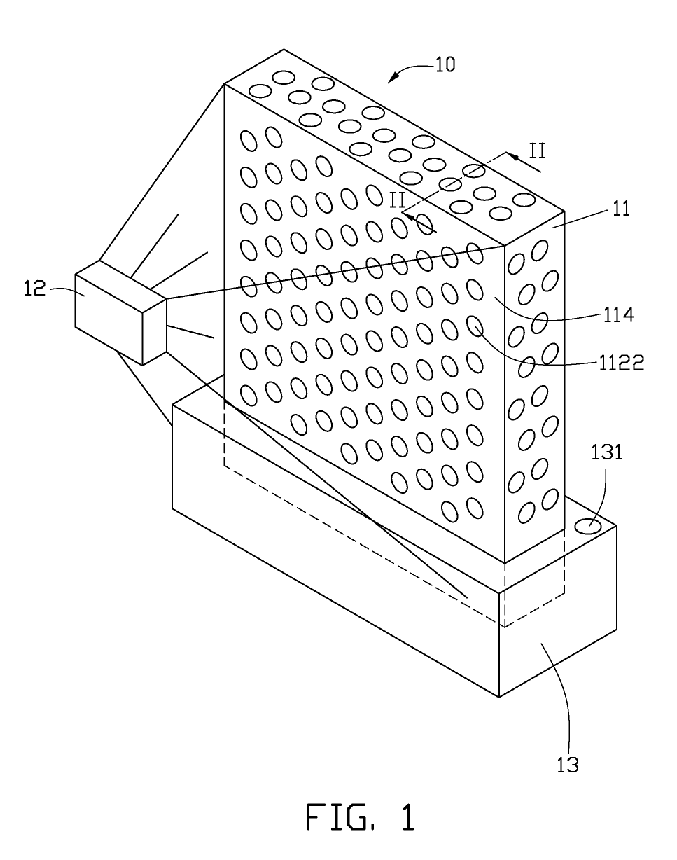

Referring to FIG. 1, a photo-catalyst air cleaner 10, in accordance with a first embodiment, comprises a photo-catalyst filter 11, a light source 12, and a water supply 13.

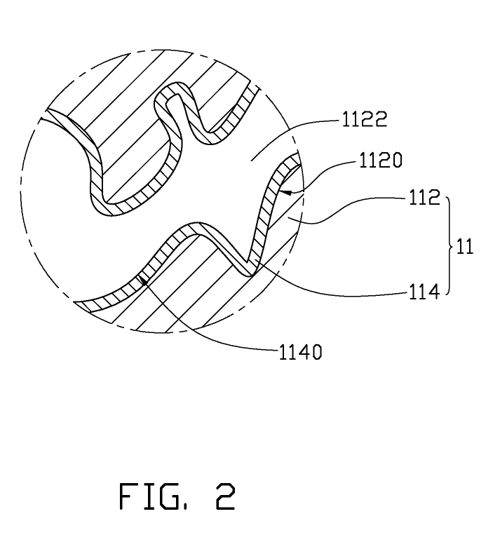

Referring to FIGS.1-2, the photo-catalyst filter 11 comprises a substrate 112 and a photo-catalyst layer 114. The substrate 112 has a surface 1120 on which the photo-catalyst layer 114 is coated by sintering, making contact with air to decompose pollutants and sterilize airborne microbes. The substrate 112 has a porous structure, including a plurality of holes 1122 defined on the surface 1120 thereof. The holes 1122 may interpenetrate or be separate from each other. The photo-catalyst layer 114 can be further coated on inner walls of the substrate 112 in the holes 1122 thereof, increasing surface area of the photo-catalyst layer 114 to improve decomposition and sterilization capability of the photo-catalyst layer 114. Preferably, the substrate 112 is alumina ceramic with high hydroscopicity. The photo-catalyst layer 114 is made of photo-catalyst material, such as tin oxide (SnO₂), zinc oxide (ZnO), tungsten oxide (WO₃), iron oxide (Fe₂O₃), SeTiO₃, cadmium selenide (CdSe), KTaO₃, cadmium sulfide (CdS) or niobium oxide (Nb₂O₅). Preferably, the photo-catalyst layer 114 is nanometer sized titanium dioxide (TiO₂) with high fluid permeability.

The light source 12 can be an ultra-violet (UV) lamp, such as a UV fluorescent lamp, and preferably a UV light-emitting diode. Additionally, the number of light sources 12 can be more than one.

The water supply 13 is a container with water with at least one hole 131 for refilling water.

In operation, one end of the photo-catalyst filter 11 is immersed in the water of the container, and a water film is formed on the surface 1140 thereof (including the substrate 112 and the photo-catalyst layer 114). The photo-catalyst layer 114 is exposed to light from the UV light emitting diode, and electron-hole pairs are excited from within the photo-catalyst layer 114 to the surface 1140 thereof, to react with the water film, forming hydroxyl radical (.OH) having higher oxidative ability. Pollutants are easily adsorbed by the water film on the surface 1140. The photo-catalyst layer 114, when excited, kills microbes and decomposes organic pollutants into water vapor (H₂O) and carbon dioxide (CO₂).

Water from the water supply 13 can be adsorbed by the surface 1140 to form hydroxyl radicals, ensuring the quality of decomposition and sterilization capability of the photo-catalyst layer 114.

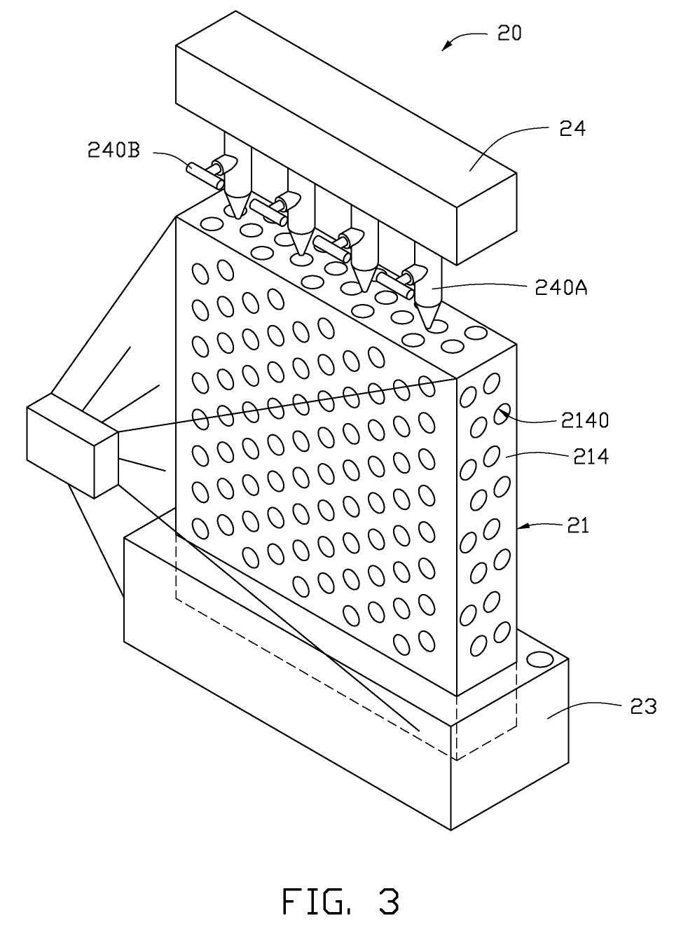

FIG. 3 shows a photo-catalyst air cleaner 20, in accordance with a second embodiment, differing from photo-catalyst air cleaner 10 of the first embodiment only in the presence of two water supplies, a first water supply 23 cooperating with a second water supply, providing water to form a water film on a surface 2140 of a photo-catalyst layer 214 to form hydroxyl radicals.

The second water supply 24 is arranged opposite the first water supply 23 at another end of the photo-catalyst filter 21, and includes a number of water outlets 240A dispensing water onto the photo-catalyst filter 21. Each of the water outlets 240A is coupled to a water valve 240B, by which the amount of water is controlled. The photo-catalyst filter 21 absorbs water from both the first and second water supplies 23, 24. It is to be understood that the second water supply 24 can also dispense water on two lateral sides of the photo-catalyst filter 21, if greater surface coverage is desired.

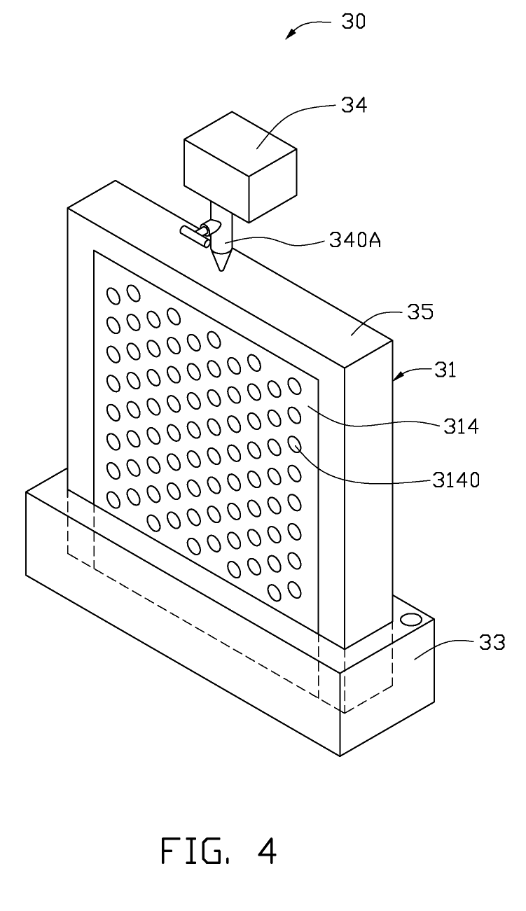

FIG. 4 shows another photo-catalyst air cleaner 30, in accordance with a third embodiment, differing from photo-catalyst air cleaner 20 of the second embodiment only in the presence of a capillary suction device 35 with high hydroscopicity, retrieving water from first and second water supplies 33, 34.

The capillary suction device 35 surrounds the photo-catalyst filter 31, and is in contact with the photo-catalyst filter 31. Water of the first and second supplies 33, 34 is absorbed by and diffused within the capillary suction device 35 quickly and uniformly. Photo-catalyst filter 31 absorbs the water via the capillary suction device 35, to form a water film on a surface 3140 of the photo-catalyst layer 314. The capillary suction device 35 allows simpler water distribution system if needed (the photo-catalyst air cleaner 30 uses only one water outlet 340A), and water film formed on the surface 3140 remains uniform and sufficient.

The capillary suction device 35 is flannelet of high hydroscopicity, adsorbent cotton, such as polyvinyl alcohol (PVA) adsorbent cotton or other fiber or fabric with high hydroscopicity.

It is believed that the present invention and its advantages will be understood from the foregoing description, and it will be apparent that various changes may be made thereto without departing from the spirit and scope of the invention or sacrificing all of its material advantages, the examples hereinbefore described merely being preferred or exemplary embodiments of the invention.

What is claimed is:

1. A photo-catalyst air cleaner, comprising
at least one light source;
a photo-catalyst filter comprising a hydrophilic substrate and a photo-catalyst layer comprising a surface on the surface of the substrate and exposed to light from the at least one light source;
a first water supply supplying water for absorption by the substrate, providing formation of a water film on the surface thereof.

2. The photo-catalyst air cleaner of claim 1, wherein the first water supply is a container with water, into which one end of the photo-catalyst filter is immersed.

3. The photo-catalyst air cleaner of claim 2, further comprising a second water supply, comprising at least one water outlet for dispensing water onto the photo-catalyst filter.

4. The photo-catalyst air cleaner of claim 3, wherein the at least one water outlet is arranged opposite to the first water supply at another end of the photo-catalyst filter.

5. The photo-catalyst air cleaner of claim 4, wherein the second water supply comprises at least one valve, to which the at least one water outlet is coupled, configured for controlling the amount of water dispensed from the water outlet.

6. The photo-catalyst air cleaner of claim 3, further comprising a capillary suction device, connected to the photo-catalyst filter to absorb water from the first and second water supplies.

7. The photo-catalyst air cleaner of claim 6, wherein the capillary suction device is flannelet or adsorbent cotton.

8. The photo-catalyst air cleaner of claim 1, wherein the base has a porous structure, a plurality of holes being defined on the surface thereof, wherein the photo-catalyst layer is further formed on inner walls of the substrate in the holes thereof.

9. The photo-catalyst air cleaner of claim 1, wherein the substrate is alumina ceramic with high hydroscopicity.

10. The photo-catalyst air cleaner of claim 1, wherein the photo-catalyst layer is titanium dioxide.

11. The photo-catalyst air cleaner of claim 1, wherein the light source is an ultraviolet light-emitting diode.
